(19) United States
(12) Patent Application Publication
(10) Pub. No.: US 2021/0054321 A1
(43) Pub. Date: Feb. 25, 2021

(54) MICROFLUIDIC DEVICES AND METHODS INCORPORATING ORGANIZED THREE-DIMENSIONAL TISSUE CONSTRUCTS

(71) Applicant: ARIZONA BOARD OF REGENTS ON BEHALF OF ARIZONA STATE UNIVERSITY, Scottsdale, AZ (US)

(72) Inventors: Mehdi Nikkhah, Scottsdale, AZ (US); Jaimeson Veldhuizen, Tempe, AZ (US)

(73) Assignee: Arizona Board of Regents on behalf of Arizona State University, Scottsdale, AZ (US)

(21) Appl. No.: 16/650,346

(22) PCT Filed: Oct. 8, 2019
(86) PCT No.: PCT/US19/55174

§ 371 Date: Mar. 24, 2020

Related U.S. Application Data
(60) Provisional application No. 62/745,683, filed on Oct. 15, 2018.

(51) Int. Cl. C12M 3/06; B01L 3/00; C12M 1/12; C12N 5/00

(52) U.S. Cl. CPC C12M 23/16; B01L 3/502715; C12M 25/14; B01L 2300/0896; B01L 2300/069; B01L 2300/0877; C12N 5/0075

(57) ABSTRACT

A microfluidic device is provided that incorporates a highly organized co-culture of live cells within a microengineered platform, by which the architecture and cellular constituents of an organ or other native tissue environment may be modeled over a long period of culture for biological and/or pharmacological studies. Vertical posts (e.g., microposts) may be used to induce alignment of hydrogel-encapsulated tissues in a cell suspension region of a microfluidic device. Such a device can complement animal studies in recapitulating pathophysiological characteristics of disease. When populated with cardiac cells, such a device provides a three-dimensional (3D) biomimetic human cardiac tissue model that enables the study of pathophysiological events involved in the transition of healthy to diseased cardiac tissue, to better inform therapeutic strategies and functional outcomes in cardiac-based therapies. Other types of cells may be used in certain embodiments. Methods of fabricating and using such microfluidic devices are also provided.

### RELATED APPLICATIONS

This application claims the benefit of U.S. Provisional Patent Application No. 62/745,383, filed Oct. 14, 2018, wherein the disclosure of such application is hereby incorporated by reference herein in its entirety.

### TECHNICAL FIELD

This disclosure relates to microfluidic devices, including devices incorporating highly organized (e.g., aligned) three-dimensional, live tissue constructs cells, and associated methods of fabricating and using such devices.

### BACKGROUND

Cardiovascular diseases persistently remain the main cause of mortality among people worldwide, despite substantial effort in research and therapeutic interventions. Cardiac ischemia, including acute myocardial infarction (MI, “heart attack”) and chronic ischemic heart disease (chronic IHD), are highly progressive biological disorders that can ultimately lead to catastrophic heart failure and death. Hypoxia has been implicated as the major regulator of ischemia-induced cardiac injury, through triggering of a multitude of molecular/cellular signaling cascades. Specifically, for MI, acute hypoxia leads to necrosis of cardiomyocytes (CMs) that induces upregulation of inflammatory cytokines and cellular paracrine signaling, followed by contractile dysfunction and adverse remodeling of the myocardium. The orchestrated crosstalk between CMs and neighboring interstitial cells (i.e., cardiac fibroblasts) adds a higher level of complexity to the mechanism of disease progression. The physiological and pathophysiological responses of the heart in healthy and diseased states have been the subject of intensive studies using gold standard animal models. However, animal models may lack physiological relevancy to humans, with the inability to precisely control micro environmental cues for cellular and molecular level studies. This has led to a major knowledge gap in full understanding of the molecular/cellular basis of cardiac injury. Furthermore, the inability of animal models to fully recapitulate human physiology often leads to failures in bench to bedside therapeutics development, highlighting the critical need to develop alternative platform technologies using human cardiac cells that would have translational advantages over animal models.

In order to fulfill the need for a physiologically relevant human heart tissue model, a majority of the research has focused on two main approaches. These approaches include the creation of three-dimensional (3D), macro-scale cardiac tissue constructs/patches, or the use of microfluidic devices to bioengineer human heart-on-a-chip models. To create 3D cardiac tissue in these models, primary CMs or stem-cell-derived cardiac cells are encapsulated in natural or synthetic hydrogels, representative of the native extracellular matrix (ECM) of myocardium. In organ-on-a-chip technology platforms, elastomers are used to create intricate microchannels and entities that house these hydrogel-encapsulated tissues. However, these approaches have yet to produce, in combination, the following items: (1) physiologically relevant anisotropic cardiac tissue, (2) appropriate selection of cell types including myocyte and non-myocytes and (3) a suitable platform, with capabilities to induce/mimic physiological conditions of a specific cardiac disease for fundamental biological discovery or drug screening. A number of cardiac tissues developed within microfluidic-based platforms form an isotropic structure or random cellular architecture, since inducing cardiac cell alignment within a 3D matrix is not a trivial task. Previous attempts have utilized micro-grooved features, to induce alignment by seeding cells on different types of polymeric or hydrogel-based substrates. Other studies have utilized microposts to guide cardiac cell organization, encapsulated in 3D hydrogel-based matrices. The few devices that do have topographical cues to induce cellular alignment fail to provide a biomimetic 3D human tissue architecture and precise mechanistic insight into the phenotypic signatures of cardiac cells and the pathophysiological alterations of myocardium from healthy to diseased state.

The other main approach that has been researched for the creation of cardiac tissue for mechanistic and biologic studies is engineering of 3D cardiac patch or tissue constructs. Recently, hydrogel-encapsulated tissue patches have utilized microscale topography, in the form of staggered microposts, to induce cardiac tissue alignment. These studies have demonstrated enhanced cardiac structure and tissue contractility, and mature gene expression, due to the environmental cues caused by the microscale topography. However, these patches tend to exist on a relatively large (i.e., macro) scale, thereby necessitating a large quantity of materials and reagents to perform the experimental procedures that would be pertinent to cardiac tissue modeling.

It would be desirable to reduce the scale of 3D tissue constructs (including but not limited to cardiac tissue constructs) to the micrometer to overcome limitations associated with conventional constructs. Current microfluidic-based models of engineered tissue fail to incorporate topographies sufficient to induce tissue alignment, and have therefore failed to recapitulate the relevant anisotropic nature of the human tissues, such as myocardial, skeletal, and neural tissues.

### SUMMARY

Disclosed herein is a microfluidic device that incorporates a highly organized co-culture of live cells within a microengineered platform, by which the architecture and cellular constituents of an organ or other native tissue environment may be modeled over a long period of culture for biological and/or pharmacological studies. Vertical posts (e.g., microposts) may be used to induce alignment of hydrogel-encapsulated tissues in a cell suspension region of a microfluidic device. Such a microfluidic device can complement animal studies in recapitulating pathophysiological characteristics of disease. When populated with cardiac cells, such a microfluidic device provides a three-dimensional (3D) biomimetic human cardiac tissue model that enables the study of pathophysiological events involved in the transition of healthy to diseased cardiac tissue, to better inform therapeutic strategies and functional outcomes in cardiac-based therapies. Other types of cells may be used in certain embodiments. Methods of fabricating and using such microfluidic devices are also provided.

In one aspect, the disclosure relates to a microfluidic device comprising: a cell suspension region; a plurality of linear arrays of vertical posts arranged within the cell suspension region; at least one hydrogel insertion port arranged upstream of the cell suspension region; at least one sample extraction port arranged downstream of the cell suspension region; a first fluid channel arranged proximate to the cell suspension region; a first fluid-permeable boundary wall arranged between the first fluid channel and the cell suspension region, and forming a first lateral boundary of the cell suspension region; a second fluid channel arranged proximate to the cell suspension region; a second fluid-permeable boundary wall arranged between the second fluid channel and the cell suspension region, and forming a second lateral boundary of the cell suspension region; a first fluid channel inlet port and a first fluid channel outlet port in fluid communication with the first fluid channel; and a second fluid channel inlet port and a second fluid channel outlet port in fluid communication with the second fluid channel; wherein the cell suspension region is enclosed from above and below.

In certain embodiments, the cell suspension region contains a 3D hydrogel matrix having cells embedded therein and arranged in contact with the plurality of linear arrays of vertical posts. In certain embodiments, the cells comprise at least one of: animal- or human-derived cardiac cells, animal- or human-derived neural cells, or animal- or human-derived skeletal muscle cells.

In certain embodiments, each vertical post of the plurality of linear arrays of vertical posts includes a tapered leading edge and a tapered trailing edge. In certain embodiments, each vertical post of the plurality of linear arrays of vertical posts includes a length, from the tapered leading edge to the tapered trailing edge, in a range of from 200 microns to 500 microns.

In certain embodiments, vertical posts of the plurality of linear arrays of vertical posts are spaced apart from adjacent vertical posts by a lengthwise dimension in a range of from 100 to 300 microns, and by a widthwise dimension in a range of from 100 to 300 microns.

In certain embodiments, the first fluid-permeable boundary wall comprises a first array of trapezoidal posts, and the second fluid-permeable boundary wall comprises a second array of trapezoidal posts. In certain embodiments, the first array of trapezoidal posts comprises a first plurality of linearly arranged trapezoidal posts, with each trapezoidal post of the first plurality of linearly arranged trapezoidal posts comprising a short end arranged closer to the cell suspension region than to the first fluid channel; and the second array of trapezoidal posts comprises a second plurality of linearly arranged trapezoidal posts, with each trapezoidal post of the second plurality of linearly arranged trapezoidal posts comprising a short end arranged closer to the cell suspension region than to the second fluid channel.

In certain embodiments, the at least one sample extraction port comprises first and second sample extraction ports that are laterally offset relative to one another. In certain embodiments, the microfluidic device further comprises a first sample extraction channel arranged between the cell suspension region and the first sample extraction port; and a second sample extraction channel arranged between the cell suspension region and the second sample extraction port; wherein the first sample extraction channel is laterally offset relative to the second sample extraction channel.

In certain embodiments, the at least one hydrogel insertion port comprises a first hydrogel insertion port and a second hydrogel insertion port that are laterally offset relative to one another; and the microfluidic device further comprises a first hydrogel insertion channel arranged between the first hydrogel insertion port and the cell suspension region, and a second hydrogel insertion channel arranged between the second hydrogel insertion port and the cell suspension region, wherein the first hydrogel insertion channel is laterally offset relative to the second hydrogel insertion channel.

In certain embodiments, the microfluidic device further comprises endothelial cells seeded in at least one of the first fluid channel or the second fluid channel.

In certain embodiments, one or more of the first fluid channel, the second fluid channel, or the cell suspension region comprises a height dimension and/or width dimension of less than 500 microns.

In certain embodiments, the microfluidic device further comprises at least one electrode arranged in conductive electrical communication with the cell suspension region.

In another aspect, the disclosure relates to a microfluidic device comprising: a cell suspension region; at least one hydrogel insertion port arranged upstream of the cell suspension region; a plurality of sample extraction ports arranged downstream of and in fluid communication with the cell suspension region, wherein each sample extraction port of the plurality of sample extraction ports is laterally offset relative to at least one other sample extraction port of the plurality of sample extraction ports; a first fluid channel arranged proximate to the cell suspension region; a first fluid-permeable boundary wall arranged between the first fluid channel and the cell suspension region, and forming a first lateral boundary of the cell suspension region; a second fluid channel arranged proximate to the cell suspension region; a second fluid-permeable boundary wall arranged between the second fluid channel and the cell suspension region, and forming a second lateral boundary of the cell suspension region; a first fluid channel inlet port and a first fluid channel outlet port in fluid communication with the first fluid channel; and a second fluid channel inlet port and a second fluid channel outlet port in fluid communication with the second fluid channel; wherein the cell suspension region is enclosed from above and below.

In certain embodiments, the microfluidic device further comprises a plurality of linear arrays of vertical posts arranged within the cell suspension region.

In certain embodiments, each vertical post of the plurality of linear arrays of vertical posts includes a tapered leading edge and a tapered trailing edge. In certain embodiments, each vertical post of the plurality of linear arrays of vertical posts includes a length, from the tapered leading edge to the tapered trailing edge, in a range of from 200 microns to 500 microns.

In certain embodiments, vertical posts of the plurality of linear arrays of vertical posts are spaced apart from adjacent vertical posts by a lengthwise dimension in a range of from 100 to 300 microns, and by a widthwise dimension in a range of from 100 to 300 microns.

In certain embodiments, the cell suspension region contains a 3D hydrogel matrix having cells embedded therein and arranged in contact with the plurality of linear arrays of vertical posts.

In certain embodiments, the first fluid-permeable boundary wall comprises a first array of trapezoidal posts, and the second fluid-permeable boundary wall comprises a second array of trapezoidal posts. In certain embodiments, the first array of trapezoidal posts comprises a first plurality of linearly arranged trapezoidal posts, with each trapezoidal post of the first plurality of linearly arranged trapezoidal posts comprising a short end arranged closer to the cell suspension region than to the first fluid channel; and the second array of trapezoidal posts comprises a second plurality of linearly arranged trapezoidal posts, with each trapezoidal post of the second plurality of linearly arranged trapezoidal posts comprising a short end arranged closer to the cell suspension region than to the second fluid channel.

In certain embodiments, the plurality of sample extraction ports comprises a first sample extraction port and a second sample extraction port; and the microfluidic device further comprises a first sample extraction channel arranged between the cell suspension region and the first sample extraction port, and a second sample extraction channel arranged between the cell suspension region and the second sample extraction port, wherein the first sample extraction channel is laterally offset relative to the second sample extraction channel.

In certain embodiments, the at least one hydrogel insertion port comprises a first hydrogel insertion port and a second hydrogel insertion port that are laterally offset relative to one another; and the microfluidic device further comprises a first hydrogel insertion channel arranged between the first hydrogel insertion port and the cell suspension region, and a second hydrogel insertion channel arranged between the second hydrogel insertion port and the cell suspension region, wherein the first hydrogel insertion channel is laterally offset relative to the second hydrogel insertion channel.

In certain embodiments, the microfluidic device further comprises endothelial cells seeded in at least one of the first fluid channel or the second fluid channel.

In certain embodiments, one or more of the first fluid channel, the second fluid channel, and the cell suspension region comprises a height dimension and/or width dimension of less than 500 microns.

In certain embodiments, the microfluidic device further comprises at least one electrode arranged in conductive electrical communication with the cell suspension region.

In another aspect, the disclosure relates to a method for fabricating a microfluidic device, the method comprising: supplying a suspension of cells in a hydrogel solution through at least one hydrogel insertion port into a cell suspension region of a microfluidic device to contact a plurality of linear arrays of vertical posts arranged within the cell suspension region, wherein the cell suspension region is enclosed from above and below, and is laterally bounded by first and second fluid-permeable boundary walls; and polymerizing the hydrogel solution within the cell suspension region to form a 3D hydrogel matrix having cells embedded therein and in contact with the plurality of linear arrays of vertical posts.

In certain embodiments, the cells embedded in the 3D hydrogel matrix comprise at least one of: animal- or human-derived cardiac cells, animal- or human-derived neural cells, or animal- or human-derived skeletal muscle cells.

In certain embodiments, the polymerizing of the hydrogel solution comprises at least one of thermal, chemical, or photonic polymerization.

In another aspect, the disclosure relates to a method for using a microfluidic device that includes a 3D hydrogel matrix having cells embedded therein contained within a cell suspension region that is laterally bounded by first and second fluid-permeable boundary walls, wherein first and second sample extraction ports are laterally offset relative to one another and arranged downstream of the cell suspension region. Such method comprises: supplying a first fluid containing at least one first component susceptible to interaction with the cells into a first fluid channel, wherein the first fluid-permeable boundary wall is arranged between the first fluid channel and the cell suspension region, to cause the at least one first component to contact cells within the 3D hydrogel matrix; supplying a second fluid containing at least one second component susceptible to interaction with the cells into a second fluid channel, wherein the second fluid-permeable boundary wall is arranged between the second fluid channel and the cell suspension region, to cause the at least one second component to contact cells within the 3D hydrogel matrix; and releasing at least some cells from the 3D hydrogel matrix to cause a first group of cells to flow through the first sample extraction port, and to cause a second group of cells to flow through the second sample extraction port.

In certain embodiments, the releasing of at least some cells from the 3D hydrogel matrix comprises enzymatic digestion of at least a portion of the 3D hydrogel matrix.

In certain embodiments, the at least one first component is compositionally different from the at least one second component.

In certain embodiments, the at least one first component comprises a different concentration than the at least one second component.

In certain embodiments, the at least one first component and the at least one second component are independently selected from the group consisting of: cytokines, growth factors, oxygen, drugs, toxins, nanoparticles, and chemical agents.

In another aspect, any one or more aspects or features described herein may be combined with any one or more other aspects or features for additional advantage. Other aspects and embodiments will be apparent from the detailed description and accompanying drawings.

### BRIEF DESCRIPTION OF THE DRAWINGS

FIG. 1 is a schematic diagram showing a microfluidic device with encapsulated cardiac tissue providing a model for healthy and diseased tissue states.

FIG. 2 is a top view of at least a portion of an example microfluidic device.

FIG. 3 is a top view of at least a portion of another example microfluidic device.

FIG. 4 is a schematic diagram showing steps of a method involving photolithography and replica molding for fabricating a microfluidic device as disclosed herein.

FIG. 5 is a perspective view of a microfluidic device according to FIG. 2.

FIG. 6 is a top plan view of a microfluidic device according to FIGS. 2 and 5 arranged proximate to a U.S. penny (one cent coin) to show the size of the microfluidic device.

FIG. 7 is a table of experimental variables that were tested with a platform to promote their optimization, with such variables including hydrogel composition, micropost shape, micropost length, widthwise post spacing, and lengthwise post spacing.

FIGS. 8-11 provide phase contrast images for microfluidic devices with different combinations of cardiomyocytes (CM): cardiac fibroblasts (CF) cell ratios and geometries for vertical posts.

FIG. 12 is a still image obtained from a contraction video for a 4:1 cell ratio of CM:CF.

FIG. 13 is a still image obtained from a contraction video for a 8:1 cell ratio of CM:CF.

FIGS. 14A-17B provide cytoskeletal staining images and their color-inverted representations of rat cardiac tissue between vertical posts within microfluidic devices according to four designs.

FIG. 18A provides a Z-stacked image of immunostained cell matrices within a microfluidic device at 20× magnification to demonstrate the three-dimensional (3D) character of the tissue within the cell suspension region.

FIG. 18B is a color-inverted representation of the Z-stacked image of FIG. 18A.

FIG. 19A provides a Z-stacked image of immunostained cell matrices within a microfluidic device at 40× magnification to demonstrate the 3D character of the tissue within the cell suspension region.

FIG. 19B is a color-inverted representation of the Z-stacked image of FIG. 19A.

FIG. 20 is a bar chart depicting calculated thickness of aligned tissues within microfluidic devices according to four designs for three experiments with triplicate samples.

FIG. 21 is a plot of orientation of 4′,6-diamidino-2-phenylindole (DAPI)-stained nuclei of cells within microfluidic devices according four designs.

FIGS. 22-25 provide images of cardiac marker staining of rat cardiac tissue between vertical posts for four designs of the microfluidic device as disclosed herein.

FIG. 26A is a 40× magnification image of a tissue within a microfluidic device according to one design.

FIG. 26B is a color inverted copy of the image of FIG. 26A.

FIG. 27 is a line graph plotting representative beating signals of a tissue for microfluidic devices according to four designs.

FIGS. 28-31B represent items obtained from preliminary studies performed on a co-culture of human induced pluripotent stem cell derived cardiomyocytes (hiPSC-CMs) and human CFs in hydrogel-encapsulated tissue within a cell suspension region of a microfluidic device as disclosed herein over a period of 6 days.

FIGS. 32A-33C represent items obtained from additional studies performed on a co-culture of hiPSC-CMs and human embryonic stem cell-derived cardiomyocytes (hESC-CMs), together referred to as human pluripotent stem cells (hPSCs), in hydrogel-encapsulated tissue within a cell suspension region of a microfluidic device as disclosed herein.

FIGS. 34A-34C provide images of expression of cardiac markers of hiPSC-derived cardiac tissues inside a microfluidic device and devices without posts.

### DETAILED DESCRIPTION

A microfluidic device disclosed herein incorporates an aligned three-dimensional (3D) tissue constructs of live cells. Such a device enables formation of a 3D biomimetic human cardiac tissue model. Other types of cells, such as neural, embryonic stem cells, or skeletal cells, may be used in certain embodiments. Vertical posts (e.g., microposts) may be provided to induce alignment of hydrogel-encapsulated tissues in a cell suspension region of a microfluidic device.

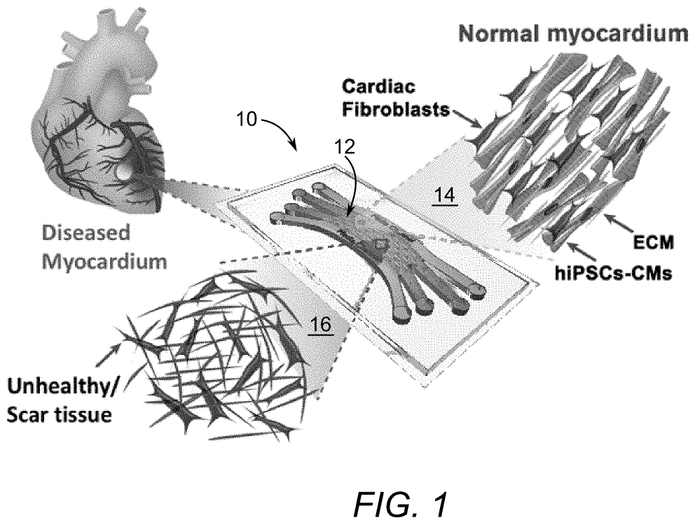

FIG. 1 is a schematic diagram showing a microfluidic device 10 (e.g., a microfluidic chip) with encapsulated cardiac tissue 12, providing a model for healthy and diseased tissue states. A first portion 14 of the encapsulated cardiac tissue 12 corresponds to a normal myocardium, with an extracellular matrix (ECM) containing cardiac fibroblasts and human induced pluripotent stem cell derived cardiomyocytes (hiPSC-CMs). A second portion 16 of the encapsulated cardiac tissue 12 corresponds to unhealthy/scar tissue contained in an ECM. The first and second portions 14, 16 of the encapsulated cardiac tissue 12 may have initially been the same in character, but diverged in health upon exposure to different conditions, growth factors, chemical agents, and/or drugs.

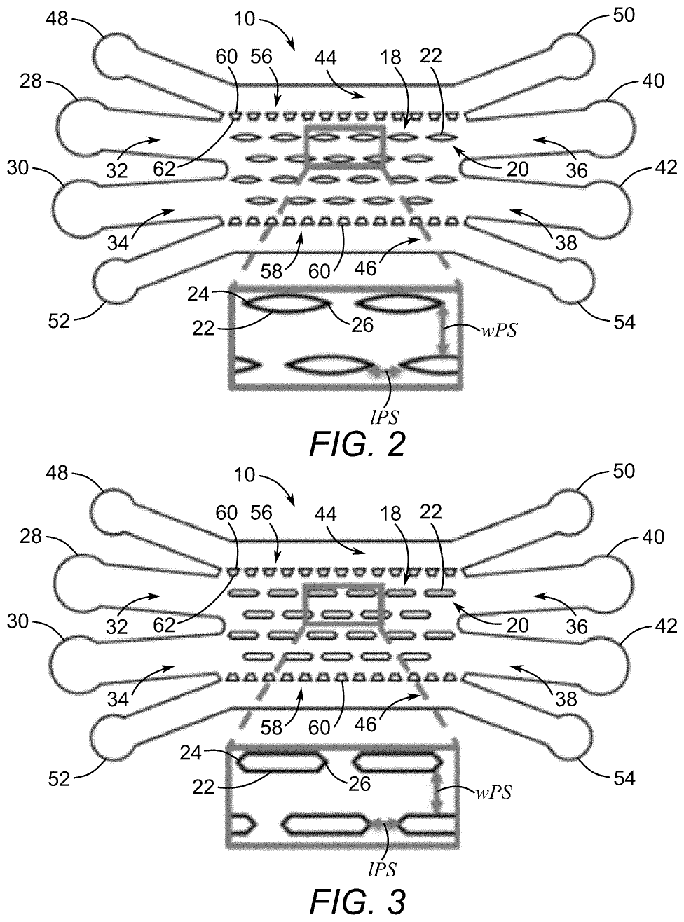

FIG. 2 is a top view of at least a portion of an example microfluidic device 10, including a cell suspension region 18 (at center) containing a plurality of (e.g., four) linear arrays 20 of vertical posts 22 each having a modified elliptical shape, with a tapered leading edge 24 and a tapered trailing edge 26. Each vertical post 22 is spaced apart from at least one adjacent vertical post 22 in a widthwise direction by a widthwise post spacing (wPS), and is spaced apart from at least one adjacent vertical post 22 in a lengthwise direction by a lengthwise post spacing (lPS). Two hydrogel insertion ports (e.g., a first hydrogel insertion port 28 and a second hydrogel insertion port 30, which are laterally offset relative to one another) lead to two laterally offset hydrogel insertion channels (e.g., a first hydrogel insertion channel 32 and a second hydrogel insertion channel 34) arranged upstream of the cell suspension region 18. Two sample extraction channels (e.g., a first sample extraction channel 36 and a second sample extraction channel 38, which are laterally offset relative to one another) lead to two sample extraction ports (e.g., a first sample extraction port 40 and a second sample extraction port 42) and are arranged downstream of the cell suspension region 18.

A first fluid channel 44 and a second fluid channel 46 are arranged along opposing sides of the cell suspension region 18. The first fluid channel 44 extends between a first fluid channel inlet port 48 and a first fluid channel outlet port 50, and the second fluid channel 46 extends between a second fluid channel inlet port 52 and a second fluid channel outlet port 54. A first fluid-permeable boundary wall 56 is arranged between the first fluid channel 44 and the cell suspension region 18, and forms a first lateral boundary of the cell suspension region 18. Similarly, a second fluid-permeable boundary wall 58 is arranged between the second fluid channel 46 and the cell suspension region 18, and forms a second lateral boundary of the cell suspension region 18. Each of the first and the second fluid-permeable boundary wall 56, 58 includes a linear array of trapezoidal posts 60, with each trapezoidal post 60 comprising a short end 62 arranged closer to the cell suspension region 18 than to the adjacent first fluid channel 44 or second fluid channel 46. In certain embodiments, each trapezoidal post 60 may be spaced apart from at least one adjacent trapezoidal post 60 by a distance of 20-30 microns (μm). The trapezoidal posts 60 ensure protection of the cell-embedded hydrogel matrix from fluidic shear stress applied by flow of fluids through the first fluid channel 44 and the second fluid channel 46, while allowing the diffusion of nutrients through the hydrogel matrix. To reduce bubble formation, the edges of the hydrogel insertion channels 32, 34 are tapered at a mouth of the cell suspension region 18.

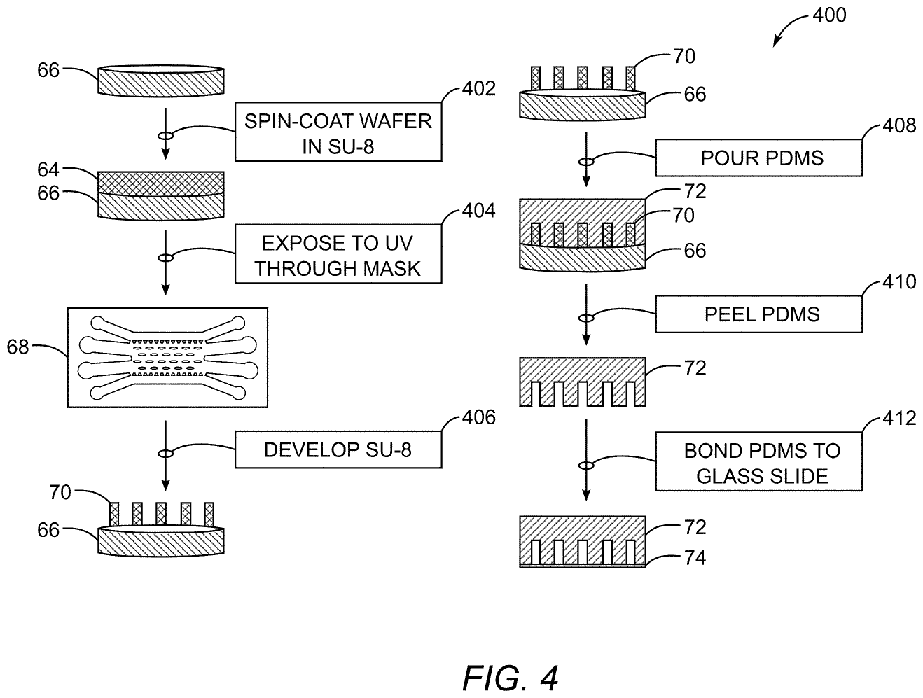

FIG. 3 is a top view of at least a portion of another example microfluidic device 10, including a cell suspension region 18 (at center) containing a plurality of (e.g., four) linear arrays 20 of vertical posts 22 each having a stretched hexagonal shape, with a tapered leading edge 24 and a tapered trailing edge 26. Other than the shape of the vertical posts 22, the microfluidic device 10 of FIG. 3 is substantially the same as the microfluidic device 10 of FIG. 2.

With reference to both FIGS. 2 and 3, in certain embodiments, each vertical post 22 may include a length, from the tapered leading edge 24 to the tapered trailing edge 26, in a range of from 200 microns to 500 μm. Moreover, in certain embodiments, each vertical post 22 may be spaced apart from at least one other adjacent vertical post 22 by a lengthwise dimension (e.g., lengthwise post spacing (lPS)) in a range of from 100 to 300 μm (or in a subrange of from 100 to 250 μm, or from 150 to 300 μm, or from 150 to 250 μm, or from 200 to 300 μm). In certain embodiments, each vertical post 22 may have a length:width ratio in a range of from about 2:1 to about 10:1, or in a subrange of from about 3:1 to about 8:1, or in a subrange of from about 4:1 to about 7:1.

With continued reference to FIGS. 2 and 3, presence of the first hydrogel insertion port 28 and the second hydrogel insertion port 30 permits hydrogel solution and cells material to be supplied to the cell suspension region 18 and distributed across a width of the cell suspension region 18. In certain embodiments, a suspension of cells in a hydrogel solution may be supplied through the hydrogel insertion ports 28, 30 to contact the linear arrays 20 of vertical posts 22 within the cell suspension region 18. Thereafter, the hydrogel solution may be polymerized within the cell suspension region 18 to form a 3D hydrogel matrix having cells embedded therein and in contact with the plurality of linear arrays 20 of vertical posts 22. Such polymerization may be accomplished by at least one of thermal, chemical, or photonic polymerization. In certain embodiments, the cells embedded in the hydrogel matrix may include at least one of: animal- or human-derived cardiac cells, animal- or human-derived neural cells, or animal- or human-derived skeletal muscle cells.

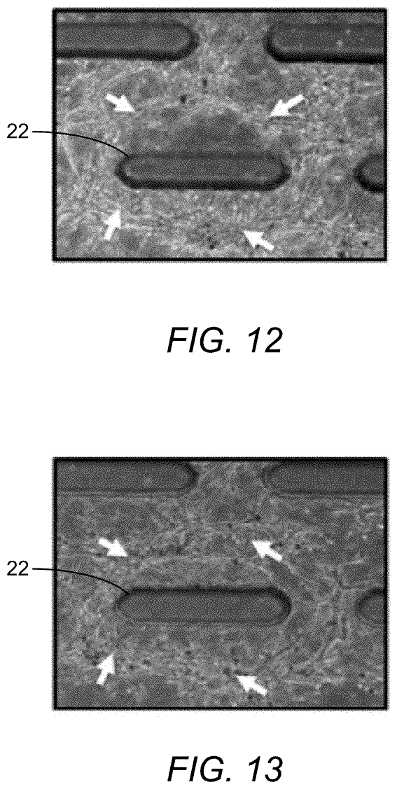

Collagen type I hydrogel, along with fibrin hydrogel, may be used for encapsulation of tissue (e.g., the cardiac tissue 12 of FIG. 1) and construction of the 3D microenvironment, with hydrogel concentration in the range as listed in FIG. 7. Other types of hydrogels known in the art, such as (but not limited to) gelatin and hyaluronic acid, could be used for establishing 3D microenvironments. For primary experiments described herein, neonatal rat cardiomyocytes (CMs) and cardiac fibroblasts (CFs) were encapsulated in a mixture of the hydrogels at a final density of 25-30×10⁶ cells/mL (at a ratio of 8:1 to 4:1). The hydrogel-encapsulated cells can be injected into the cell suspension region 18 through the hydrogel insertion ports 28, 30 (definable by coring through polydimethylsiloxane (PDMS) of the microfluidic device 10) and associated hydrogel insertion channels 32, 34. Following gel injection, a microfluidic device 10 may be placed in an incubator for 30 minutes at 37° C. in a 5% CO₂ atmosphere to allow for gel polymerization. Thereafter, the first fluid channel 44 and the second fluid channel 46 may be filled with media for passage through the fluid-permeable boundary walls 56, 58 to permit nutrients to diffuse through the tissue.

With continued reference to FIGS. 2 and 3, presence of the first fluid channel 44 and the second fluid channel 46 proximate to lateral boundaries of the cell suspension region 18 permits different fluids to be supplied to the first fluid channel 44 and the second fluid channel 46, respectively, wherein such fluids may diffuse through the fluid-permeable boundary walls 56, 58 to interact with different portions of the cells suspended in the cell suspension region 18. “Different fluids” in this context refers to fluids that may differ in composition and/or concentration. Optionally, fluids may be supplied at different flow rates and adjusted shear stresses to the first fluid channel 44 and the second fluid channel 46. When different fluids are simultaneously supplied to the first fluid channel 44 and the second fluid channel 46, a gradient condition of one or more constituents of such fluids may result in the cell suspension region 18. In this manner, cells located at different locations (e.g., closer to the first fluid channel 44 than the to the second fluid channel 46) within the cell suspension region 18 may be exposed to different conditions. This may be used to assess whether and how differing presence and/or concentration of one or more constituents of fluids supplied to the first fluid channel 44 and the second fluid channel 46 may affect condition of cells within the cell suspension region 18.

The existence of two laterally offset sample extraction channels 36, 38 and sample extraction ports 40, 42 downstream of the cell suspension region 18 also permits cells subjected to different conditions to be separately extracted from the microfluidic device 10 to permit such cells to be separately analyzed. For example, in certain embodiments, a method utilizing the microfluidic device 10 of either FIG. 2 or FIG. 3 may include: supplying a first fluid containing at least one first component susceptible to interaction with the cells into the first fluid channel 44, to cause the first fluid to permeate the first fluid-permeable boundary wall 56 and cause at least one first component to contact cells within the 3D hydrogel matrix (e.g., in a portion of the cell suspension region 18 closer to the first fluid channel 44); and supplying a second fluid containing at least one second component susceptible to interaction with the cells into the second fluid channel 46, to cause the second fluid to permeate the second fluid-permeable boundary wall 58 and cause the at least one second component to contact cells within the 3D hydrogel matrix (e.g., in a portion of the cell suspension region 18 closer to the second fluid channel 46). In certain embodiments, the first fluid and the second fluid may be independently selected from the group consisting of: saline, cell culture media, air, plasma, serum, whole blood, buffers, and cytokines. In certain embodiments, the at least one first component and the at least one second component may be independently selected from the group consisting of: cytokines, growth factors, oxygen, drugs, toxins, nanoparticles, and chemical agents. After fluids are supplied to the first fluid channel 44 and the second fluid channel 46 for a desired period of time, at least some cells may be released from the 3D hydrogel matrix to cause a first group of cells to flow through the first sample extraction port 40, and to cause a second group of cells to flow through the second sample extraction port 42. Such releasing may include, for example, enzymatic digestion of at least a portion of the 3D hydrogel matrix, which may be accomplished by supplying appropriate enzymes to the first fluid channel 44 and/or the second fluid channel 46.

The design of the microfluidic device 10 was performed using AutoCAD based on the following dimensions. The dimensions of the length, width, and height of the fluid channels 44, 46 are 4-6 millimeters (mm), 750-1000 μm, and 200-300 μm respectively. The total width of the cell suspension region 18 is 2-3 mm. Within the cell suspension region 18, microposts (e.g., the vertical posts 22) are localized with experimentally defined geometrical features to optimize 3D cellular alignment, while maintaining cellular connectivity and paracrine signaling. In certain embodiments, these vertical posts 22 may be in the range of 500-800 μm long, 100-200 μm wide, and spaced with lengthwise post spacing (lPS) of 150-350 μm and widthwise post spacing (wPS) of 150-300 μm.

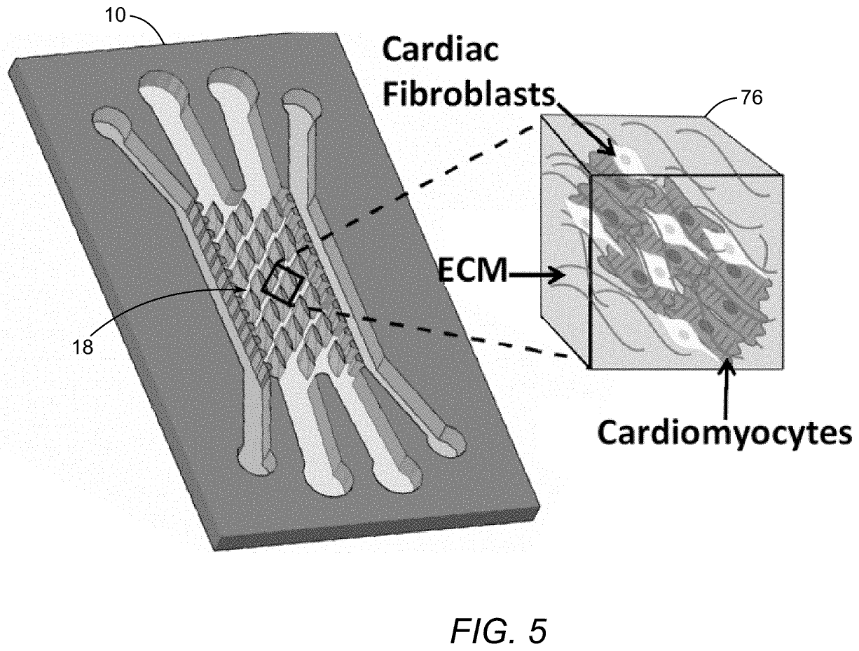

FIG. 4 is a schematic diagram showing steps of a method 400 involving photolithography and replica molding for fabricating a microfluidic device 10 as disclosed herein. Microfluidic devices 10 as disclosed herein may be fabricated with PDMS using SU-8 photolithography and replica molding technique. The method starts at operation 402, with spin coating SU-8 photoresist 64 on a silicon wafer 66 to a thickness of 200 μm. Then, at operation 404 the coated wafer 66 is exposed to ultraviolet (UV) light through a transparent mask 68 with the microfluidic device 10 design, created using AutoCAD (minimum features of 20 um). At operation 406, the SU-8 photoresist 64 is developed, resulting in a negative replica 70 of the microfluidic device 10. This negative replica 70 may be used to produce a positive replica 72 from the PDMS.

The wafer 66 is prepared for PDMS replica molding via silanization (e.g., using methyltryichlorosilane (MTCS)) of the surface to reduce attraction between the cast PDMS and the SU-8 features. The PDMS to be cast is prepared through mixing of the base to curing agent at a ratio of 10:1. At operation 408, this solution is poured over the silanized silicon wafer 66, then degassed in a vacuum and baked in an oven for 2 hours at 80° C. After polymerization, at operation 410 the PDMS is peeled from the wafer 66 and, in examples where an array of microfluidic devices 10 are molded together, each microfluidic device 10 in the array is cut. The inlet and outlet ports are cored down to the channels using standard 1-2 mm punches. For example, the first fluid channel inlet port 48 and first fluid channel outlet port 50 are cored down to the first fluid channel 44, and the second fluid channel inlet port 52 and second fluid channel outlet port 54 are cored down to the second fluid channel 46. In some examples, a similar process is applied for the hydrogel insertion ports 28, 30 and/or the sample extraction ports 40, 42. At operation 412, the PDMS surfaces of the positive replica 72 are rendered hydrophilic through the use of air plasma, then bonded to glass slides 74 to create microfluidic channels (e.g., the fluid channels 44, 46, the cell suspension region 18, the hydrogel insertion channels 32, 34, and the sample extraction channels 36, 38). The PDMS microfluidic channel platform is then sterilized at 120° C. for 20 minutes in a wet cycle, followed by a dry cycle at 120° C. for 35 minutes.

In certain embodiments, one or more electrodes may be provided on at least one surface of a substrate, and such electrode(s) may be in conductive electrical communication with an interior of the microfluidic device 10 (e.g., with the cell suspension region 18). In certain embodiments, one or more electrodes may be used to stimulate tissue (e.g., stimulate beating of cardiac cells). In certain embodiments, one or more electrodes may be used for reading or sensing signals generated by the tissue.

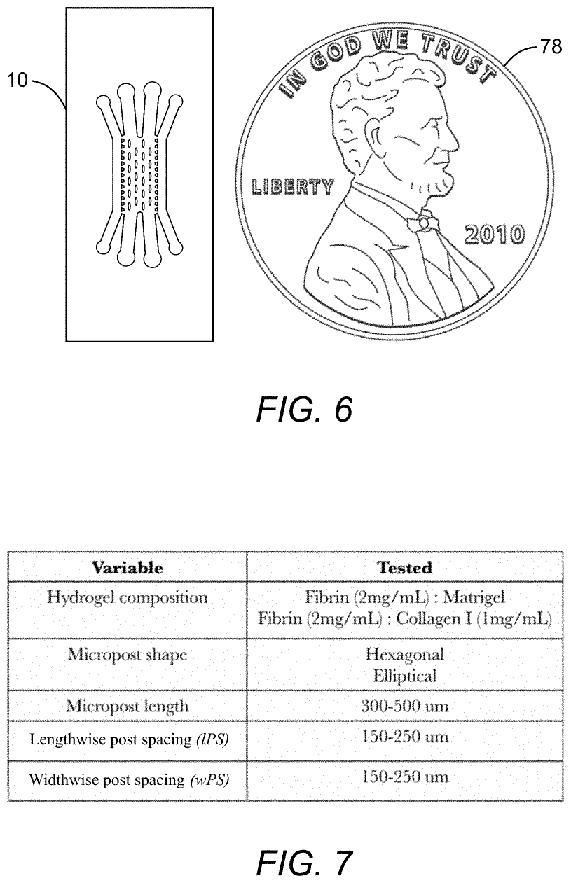

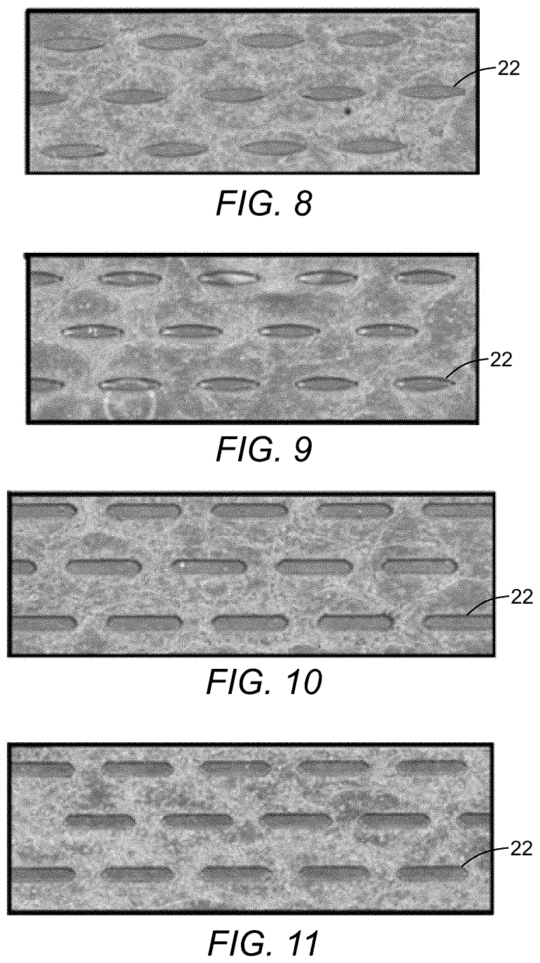

FIG. 5 is a perspective view of a microfluidic device 10 according to FIG. 2, showing the cell suspension region 18 containing a 3D hydrogel matrix 76 (i.e., a Fibrin:Collagen I hydrogel) forming an ECM that contains CFs and CMs, alongside the bordering first fluid channel 44 and second fluid channel 46. FIG. 6 is a top plan view of a microfluidic device 10 according to FIGS. 2 and 5 arranged proximate to a U.S. penny 78 (one cent coin) to show the size of the microfluidic device 10.

Microfluidic devices 10 as disclosed herein have been tested and analyzed to determine their utility and feasibility in creating 3D biomimetic cardiac tissue. Initial studies involved use of neonatal rat CMs and CFs, isolated by previously established procedures, encapsulated within a Collagen/Fibrin hydrogel and maintained within a cell suspension region 18 of a microfluidic device 10 for an extended 14-day culture period, while maintaining high cell survival. These experiments allowed for optimization of various parameters, such as viability, alignment, contraction, and cardiac marker expression. Specifically, varying cell ratios and compositions of hydrogels were studied to enhance tissue level alignment and viability of the cardiac tissue. FIG. 7 is a table of experimental variables that were tested with the microfluidic devices 10 to promote their optimization, with such variables including hydrogel composition, micropost shape, micropost length, widthwise post spacing (wPS), and lengthwise post spacing (lPS) (e.g., for the vertical posts 22 of FIGS. 2 and 3).

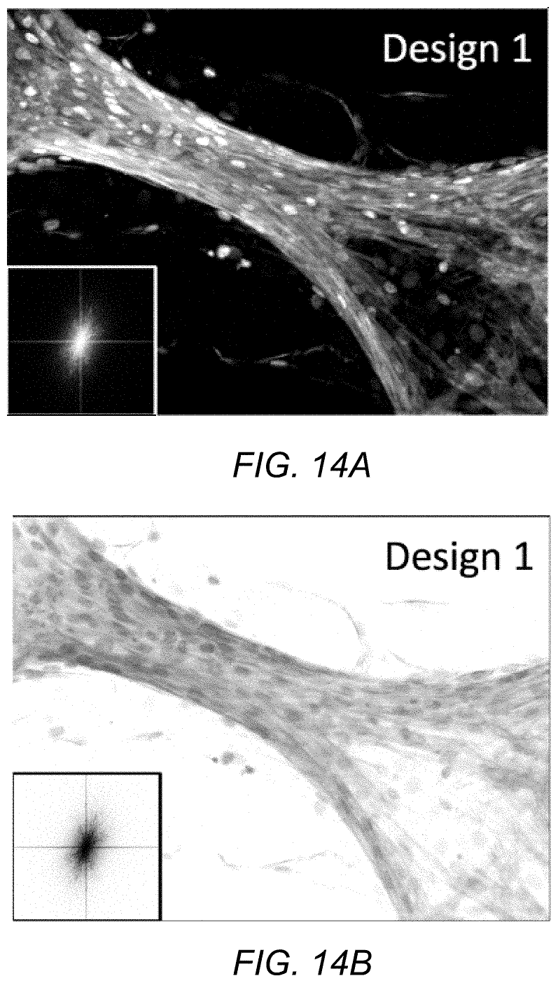

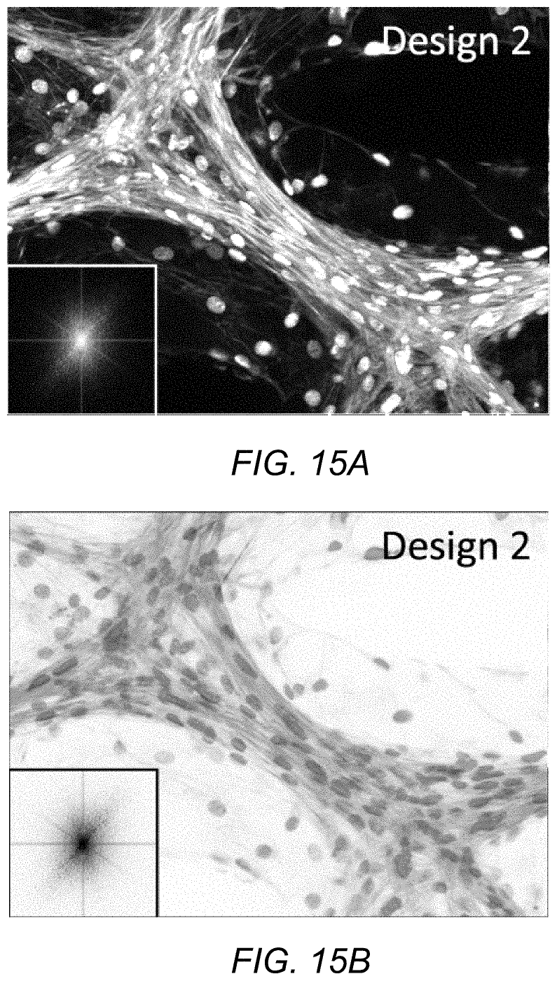

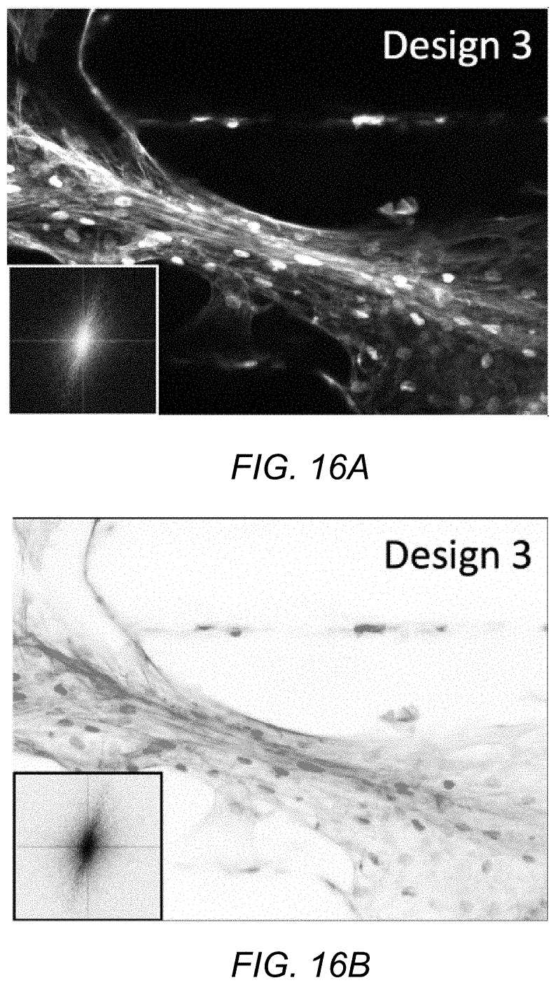

FIGS. 8-11 provide phase contrast images for microfluidic devices 10 with different combinations of CM:CF cell ratios and geometries for the vertical posts 22. In particular, FIG. 8 represents a 8:1 CM:CF cell ratio and a modified elliptical geometry for the vertical posts 22; FIG. 9 represents a 4:1 CM:CF cell ratio and a modified elliptical geometry for the vertical posts 22; FIG. 10 represents a 8:1 CM:CF cell ratio and a stretched hexagonal geometry for the vertical posts 22; and FIG. 11 represents a 4:1 CM:CF cell ratio and a stretched hexagonal geometry for the vertical posts 22. FIG. 12 is a still image obtained from a contraction video for the 4:1 cell ratio of CM:CF, and FIG. 13 is a still image obtained from a contraction video for the 8:1 cell ratio of CM:CF.

Through the performance of preliminary studies, more synchronous cardiac tissue contraction and higher viability were observed during the extended culture for the 4:1 CM:CF cell ratio, over the 8:1 CM:CF cell ratio. Specific studies into microfluidic device 10 and micro-feature dimensions have demonstrated, through F-actin staining, an optimized degree of cardiac tissue alignment for the platform with elongated topography and narrow gap widths. Additionally, the platform has also demonstrated its utility in culturing hiPSC-CMs.

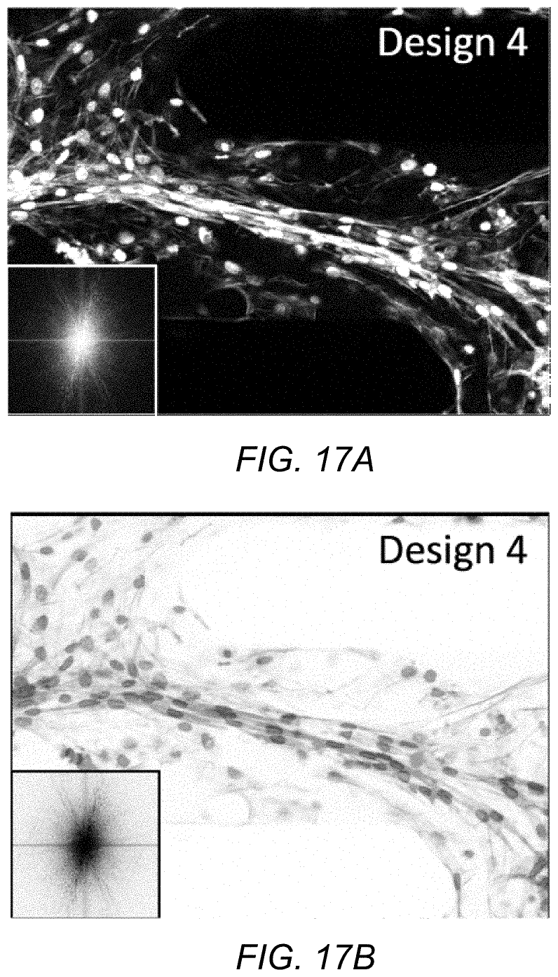

FIGS. 14A, 15A, 16A, and 17A provide cytoskeletal staining images of rat cardiac tissue between the vertical posts 22 within microfluidic devices 10 according to four designs based on FIGS. 2 and 3, Design 1, Design 2, Design 3, and Design 4 respectively. All designs incorporate vertical posts 22 with a 500 μm length and a 100 μm width, with lengthwise post spacing (lPS) of 150 μm. The geometry of the vertical posts 22 in Designs 1 and 2 (FIGS. 14A and 15A) is modified elliptical, whereas the geometry of the vertical posts 22 in Designs 3 and 4 (FIGS. 16A and 17A) is stretched hexagonal. The widthwise post spacing (wPS) of Designs 1 and 3 (FIGS. 14A and 16A) is 200 um, and the widthwise post spacing (wPS) of Designs 2 and 4 (FIGS. 15A and 17A) is 150 um. 4′,6-diamidino-2-phenylindole (DAPI) is represented in brighter spots and F-actin is represented in gray. In FIGS. 14A, 15A, 16A, and 17A, the inset images each represent a fast Fourier transform (FFT) of the actin stain. FIGS. 14B, 15B, 16B, and 17B are color-inverted representations of the cytoskeletal staining images of 14A, 15A, 16A, and 17A, respectively.

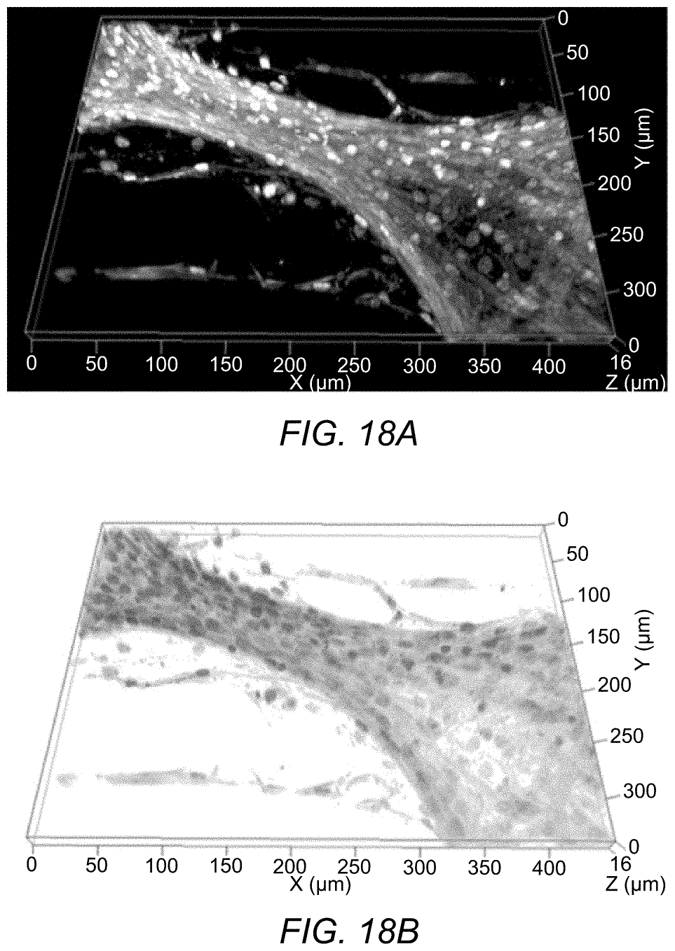

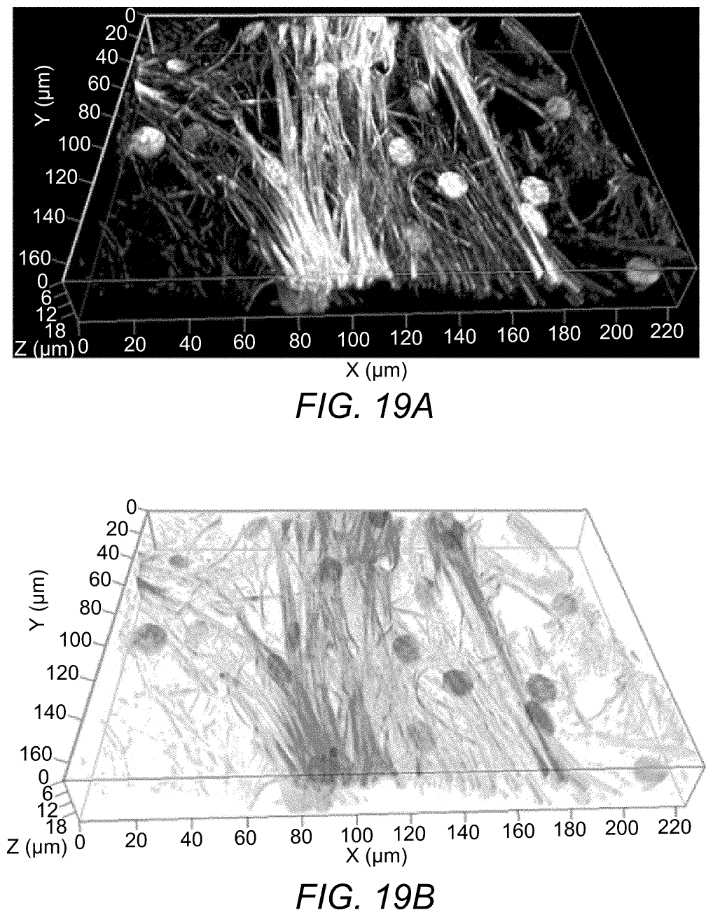

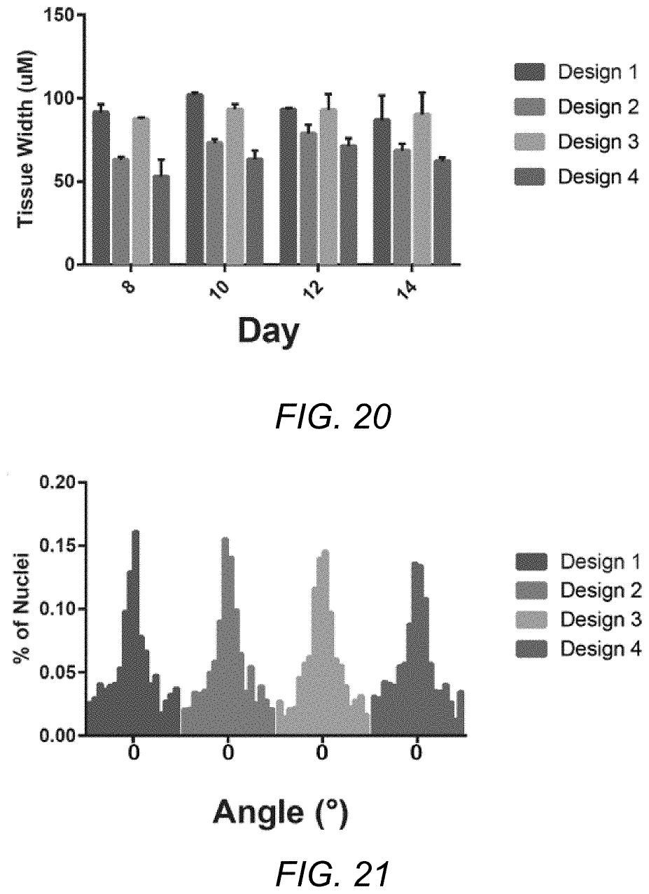

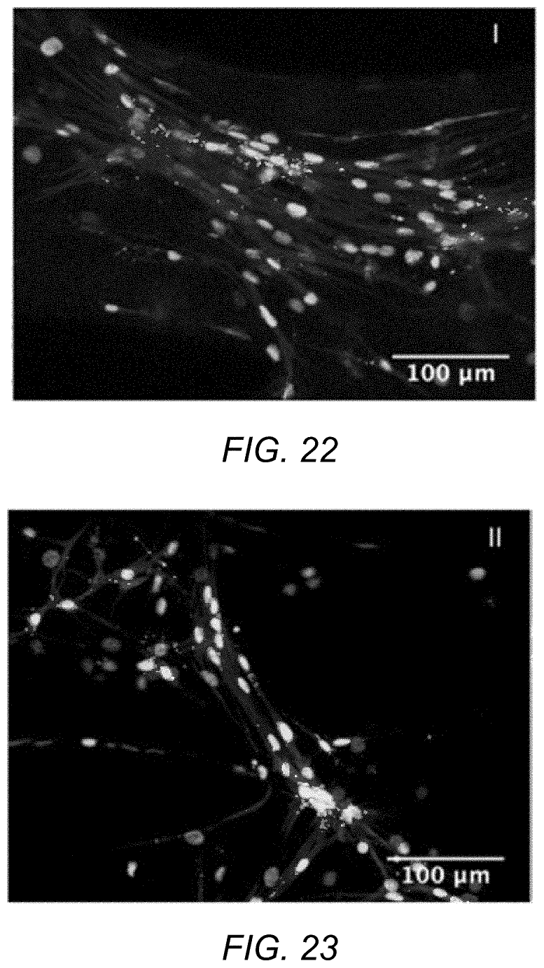

FIGS. 18A and 19A provide Z-stacked images of immunostained cell matrices within a microfluidic device 10 according to Design 1 at 20× and 40× magnification, respectively, to demonstrate the 3D character of the tissue within the cell suspension region 18. FIGS. 18B and 19B are color-inverted representations of the Z-stacked images of FIGS. 18A and 19A, respectively.

To analyze cardiac tissue thickness, phase contrast images of samples captured on Days 8, 10, 12, and 14 were used. Images were processed using the NIH ImageJ and the thickness of the tissue was measured by drawing a line across the tissue perpendicular to the alignment axis to yield the thickness. For the analysis, values were obtained over a course of 3 independent experiments, and each experiment had 2-3 technical replicates.

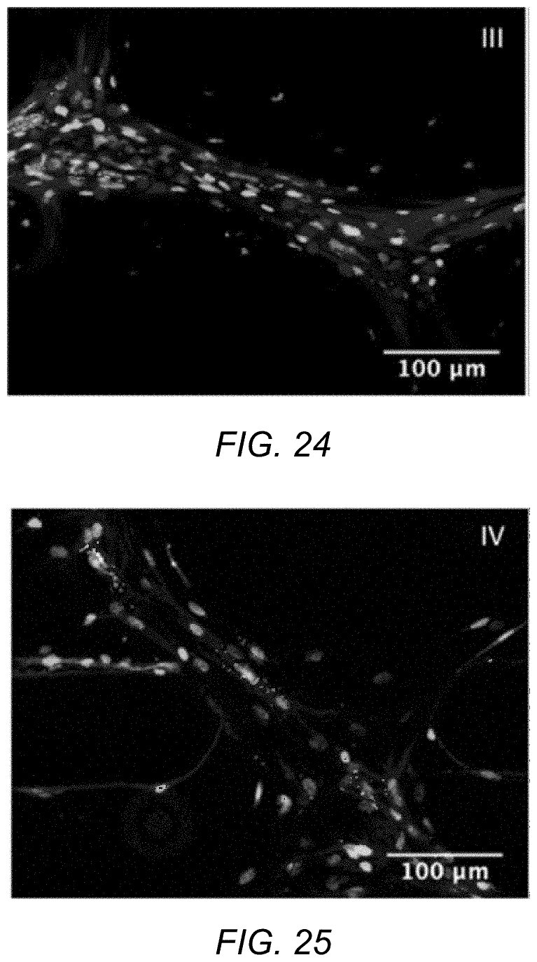

FIG. 20 is a bar chart depicting calculated thickness of aligned tissues within microfluidic devices 10 according to Designs 1 to 4 (arranged sequentially from left to right), for three experiments with triplicate samples. The specific dimensionalities of the microfluidic device 10 according to Design 1 were capable of maintaining thicker aligned tissue than microfluidic devices 10 according to Designs 2-4, demonstrating the effectiveness of Design 1 in mimicking anisotropy found in native heart tissue architecture.

Cell orientation analysis was performed through FFT analysis of actin-stained images of the cardiac tissue within a microfluidic device 10 as disclosed herein (e.g., according to Design 3). After binary thresholding of the FFT graph, lengths of the major and minor axes were measured. Then, using a previously established equation¹, the orientation index was calculated Nichol J W, Engelmayr G C, Cheng M, Freed L E. Co-culture induces alignment in engineered cardiac constructs via MMP-2 expression. Biochemical and biophysical research communications. 2008; 373(3):360-365. doi:10.1016/j.bbrc.2008.06.019 for each microfluidic device 10 and averaged, to provide an orientation index value. An orientation index value of 0.566 was calculated for a microfluidic device 10 as disclosed herein.

The alignment of cardiac tissue within the cell suspension region 18 may be assessed through staining of the cytoskeletal marker, F-actin, and cell nuclei, DAPI, to quantify cellular alignment after two weeks in culture. For immunostaining, the samples may be fixed on Day 14 in 4% (v/v) paraformaldehyde (PF) solution in Dulbecco's phosphate-buffered saline (DPBS), and nuclei of the cells may be tagged with DAPI while F-actin cytoskeletal fibers may be stained with Alexa Fluor 488 phalloidin. These immunostained images may be measured in NIH ImageJ software for FFT of the F-actin stain, and to quantify nuclei alignment through DAPI staining. Additionally, phase contrast images can be recorded throughout culture to measure varying thickness of the cultured tissue. These analyses will determine the optimal microfluidic device 10 dimensions for cardiac tissue culture and alignment.

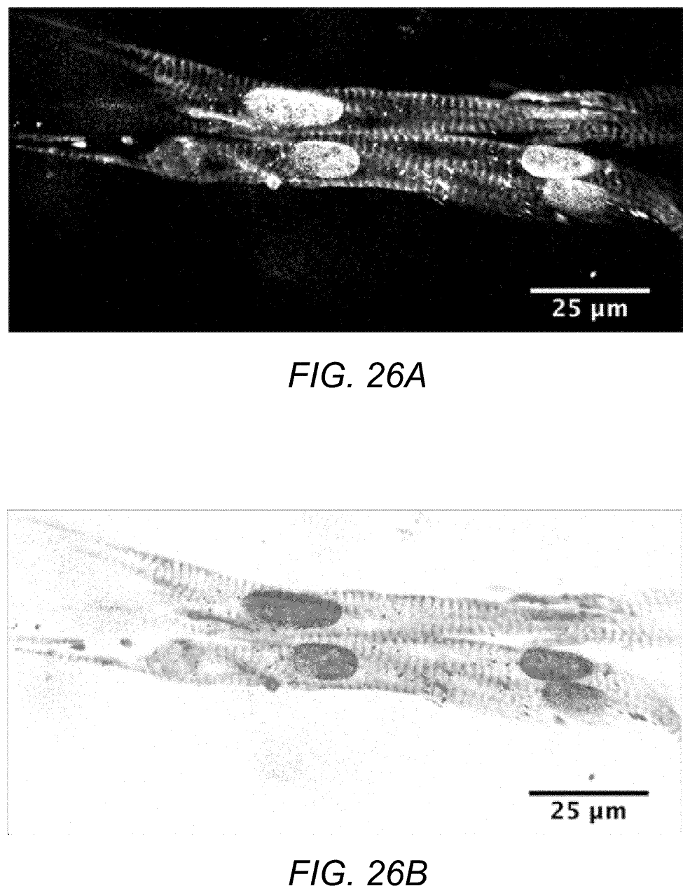

FIG. 21 is a plot of orientation of DAPI-stained nuclei of cells within microfluidic devices 10 according to Designs 1 to 4. Images were oriented so the alignment axis is 0°, as designated by tick marks along x-axis, with each bar representing a 10° deviation. As shown in FIG. 21, cells within microfluidic devices 10 according to Design 1 exhibit the highest degree of nuclei orientation.

To confirm proper expression of cardiac markers, cardiac tissue within the cell suspension region 18 may be assessed through staining of the cardiac markers sarcomeric alpha actinin and connexin 43. The relative cardiac marker expression will determine optimal cell culture ratio and period of experimental culture. Additionally, videos of duration of thirty seconds can be recorded via phase microscopy of the cultured tissue to measure spontaneous beating at different time periods of culture.

Phase contrast and fluorescence images were acquired using Zeiss Axio Observer Z1 equipped with Aptoome2 (Zeiss) and ZenPro software. Throughout cell culture period, samples were imaged every other day through phase contrast in 10× objective. Immunofluorescent images were taken at Day 14 using 10×, 20×, and 40× objectives and Z-stacked images were captured and reconstructed to form representative 3D images. Time-lapse imaging of the samples was completed on Days 8, 10, 12, and 14 to capture spontaneous contraction of the tissue. Movies were recorded at 10× objective for 30 seconds.

To analyze cardiac tissue alignment, fluorescent images of samples, stained for F-actin and DAPI, captured on Day 14, were used. Images were processed using the NIH ImageJ and the orientations of the nuclei were extracted via the Analyze Particle plugin. For the analysis, two independent experiments were used, and each experiment had 2-3 technical replicates.

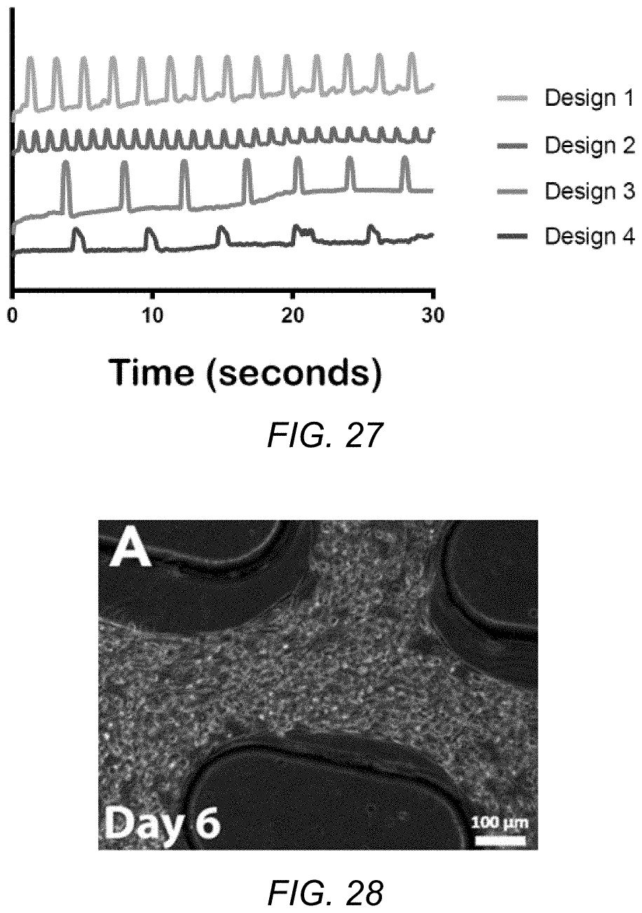

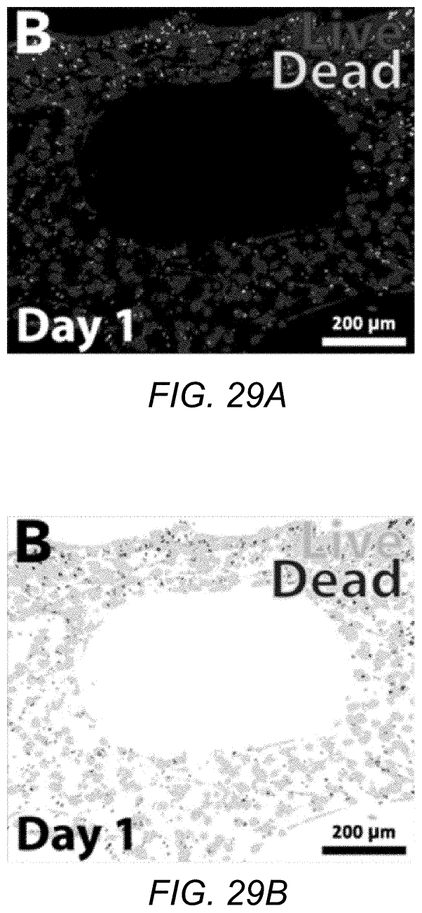

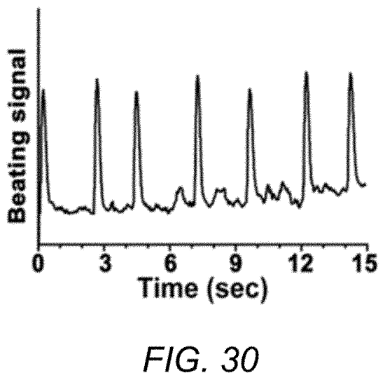

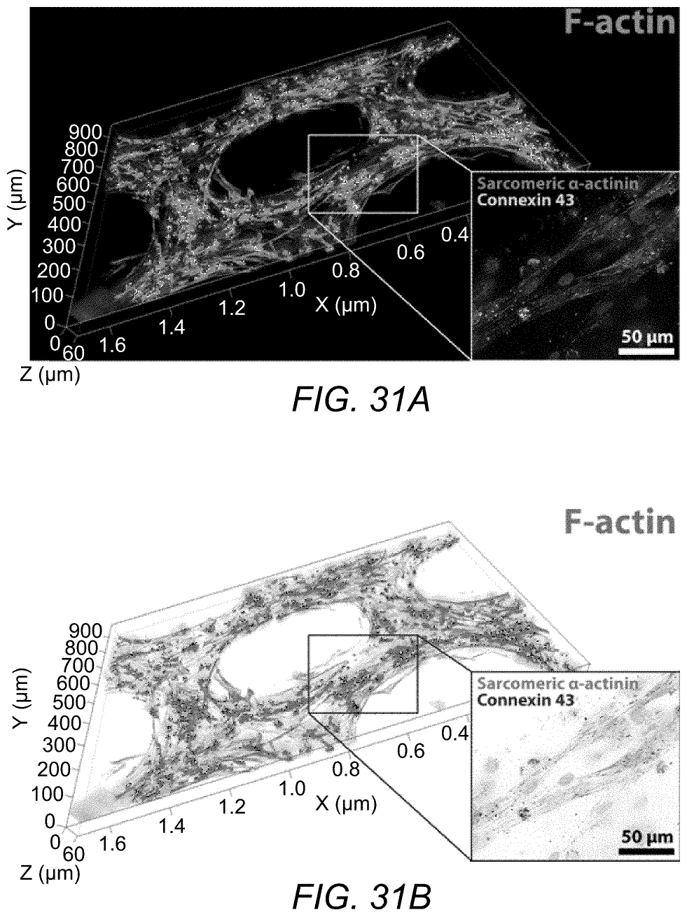

FIGS. 22-25 provide images of cardiac marker staining of rat cardiac tissue between vertical posts 22 for Design 1 to Design 4 of the microfluidic device 10 as disclosed herein. In such figures, the brightest spots are DAPI, medium gray areas are connexin 43, and the darkest gray is sarcomeric alpha-actinin. FIG. 26A is a 40× magnification image of the tissue within a microfluidic device 10 according to Design 1. FIG. 26B is a color inverted copy of the image of FIG. 26A. FIG. 26A shows striated sarcomeres and abundant expression of cardiac gap junctions. FIG. 27 is a line graph plotting representative beating signals of the tissue for microfluidic devices 10 according to Design 1 to Design 4 (arranged sequentially from top to bottom). Tissue cultured within microfluidic devices 10 according to all four designs exhibited consistent spontaneous beating, demonstrating viability of the tissue.

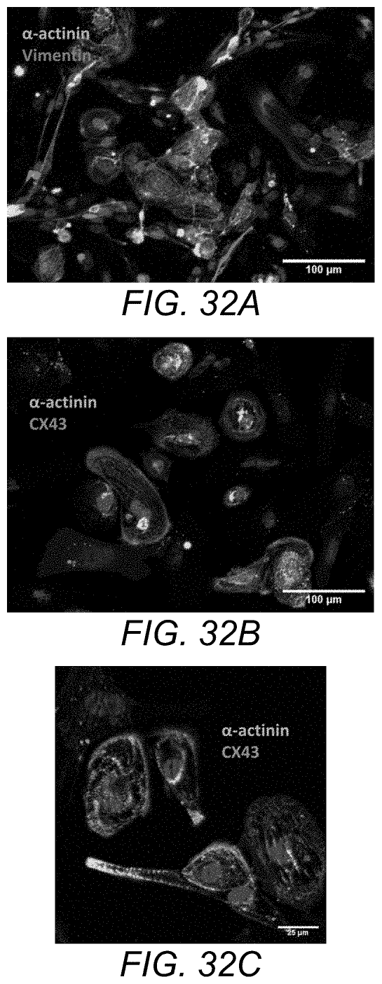

FIGS. 28-31B represent items obtained from preliminary studies performed on a co-culture of hiPSC-CMs and human CFs in hydrogel-encapsulated tissue within a cell suspension region 18 of a microfluidic device 10 as disclosed herein over a period of 6 days. FIG. 28 is a phase contrast image, taken at day 6, of tissue between vertical posts 22 of the microfluidic device 10. FIG. 29A is a cardiac marker stained image of the tissue surrounding a single vertical post 22 taken at day 1, with darker gray depicting live cells, and brighter spots depicting dead cells. FIG. 29B is a color inverted copy of FIG. 29A. FIG. 30 is a plot of a representative beating signal showing seven beats at regular intervals within a fifteen second period. FIG. 31A is a cardiac marker stained image showing organization of actin fibers between vertical posts 22 of the microfluidic device 10, with an inset portion showing cardiac specific proteins sarcomeric alpha actinin and connexin 43. FIG. 31B is a color inverted copy of FIG. 31A.

Many iterations of the microfluidic device 10 were created and experimentally tested to analyze the effect of varying dimensions of the microfluidic device 10 on alignment of the encapsulated cardiac tissue 12. A variety of lengths of the vertical posts 22, widthwise post spacings (wPS), and lengthwise post spacings (lPS) were experimentally tested through cardiac tissue culture and subsequent cytoskeletal staining. Once a length range of 300 μm-500 μm, width range of 100 μm-250 μm, and lengthwise post spacing (lPS) range of 150 μm-250 μm for the vertical posts 22 were determined, the final variables to optimize were the widthwise post spacing (wPS) and the geometry of the vertical posts 22. FIGS. 8-13 show the immunostaining results of the microfluidic devices 10 with the aforementioned dimensions, with widthwise post spacing (wPS) of 150 and 200 um (e.g., falling within a range of range of 150 μm-250 μm), and geometries of the vertical posts 22 as elliptical and hexagonal. Additionally, the effect of microfluidic device 10 dimensions on tissue contraction and cardiac marker expression was analyzed. The microfluidic device 10 with optimized tissue thickness, contraction, and alignment was experimentally determined to be Design 1, with widthwise post spacing (wPS) of 200 um and elliptical geometry for the vertical posts 22.

For immunofluorescence staining, the hydrogel-encapsulated cells within the microfluidic device 10 were fixed in 4% paraformaldehyde (PFA). The microfluidic devices 10 were kept in an incubator (humidified, 37° C., and 5% CO₂) for 15 minutes. Afterward, the cells were rinsed with PBS-glycine 2× for 10 minutes of incubation each at room temperature. The final wash was with PBS-Tween-20 ((PBS-Polyoxyethylene (20) sorbitan monolaurate) (0.05% (v/v) Polyoxyethylene (20) sorbitan monolaurate in PBS) for 10 minutes at room temperature. Then, the cells were permeablized with 0.1% Triton-X-100 for 30 minutes at room temperature. To inhibit non-specific binding of the antibodies, blocking was then performed with 10% goat serum solution for one hour at room temperature. To stain for cardiac markers, the primary antibodies for sarcomeric alpha actinin and connexin 43 were diluted in 10% goat serum, and centrifuged at 14,000 RPM for 10 minutes. Then, these antibodies were applied to the samples and kept at 4° C. overnight. The following day, the samples were washed with PBS-Tween-20 three times for 20 minutes each at room temperature. Then, the secondary antibodies were applied. After 30 minutes to 1 hour, the samples were washed with PBS-Tween-20 three times for 20 minutes each at room temperature. To stain for the actin cytoskeleton and the nucleus, Alexa Fluor488 Phalloidin and DAPI were added to the samples and left at 4° C. overnight. Then the samples were washed with PBS-Tween-20 three times for 20 minutes each at room temperature. Finally, the samples were imaged using fluorescence microscopy (Zeiss Axio Observer Z1 with the Zen Pro software suite) equipped with Apotome.2

For the primary feasibility studies, the experiments were performed using hiPSC-CMs obtained from a commercial vendor (e.g., Cellular Dynamics International (CDI)) to be readily differentiated from hiPSCs based on standard protocols. To culture, the frozen vial of hiPSC-CMs may be thawed into the CDI Plating Medium, and then the cells may be maintained in the CDI Maintenance Medium.

On the other hand, for differentiation of hiPSCs toward CMs, first hiPSCs are cultured in Geltrex coated plates. To coat plates with Geltrex, 120 uL of Geltrex is thawed into 12 mL of DMEM/F-12K. Then 2 mL of this suspension is plated into each well of a 6-well plate. The plate is left in the incubator at 37° C. for at least an hour. Then, the media is aspirated and the plate is ready to use for cell culture.

Upon thawing and passaging, the E8 media for hiPSC culture is modified to contain 10 uM of Thiazovivin for 24 hours of culture, then the media is changed to E8 media. For routine media changes, just E8 media is used for hiPSC culture, with media changes every day.

To create a co-culture, the CFs (either rat- or human-derived) need to be dissociated from their culture flask. To do so, aspirate media, wash the vessel with 1×DPBS, then add Trypsin 1× and incubate at 37° C. for −5 minutes until the cells begin to round up. Then wash the vessel with complete DMEM (DMEM+10% FBS+1% Pen/strep+1% L-glutamine). Then collect the cell suspension and count via hemocytometer to determine volume resuspension for desired cell encapsulation density. Then, the cells are centrifuged at 200 g for 5 minutes. The supernatant is aspirated, then the cell pellet is resuspended in predetermined volume of complete DMEM. The CMs and the CFs are mixed together at the desired ratio (4:1), then mixed with 2 mg/mL fibrinogen, thrombin at 1 U/mg of fibrinogen, and 1 mg/mL collagen.

Preliminary studies with hiPSC-CMs in co-culture with human CFs have demonstrated the formation and assembly of human-specific 3D myocardium with highly organized architecture within microfluidic devices 10 disclosed herein. Such microfluidic devices 10 have demonstrated their utility in establishing a 3D cardiac tissue of both primary animal- and stem cell-derived CMs.

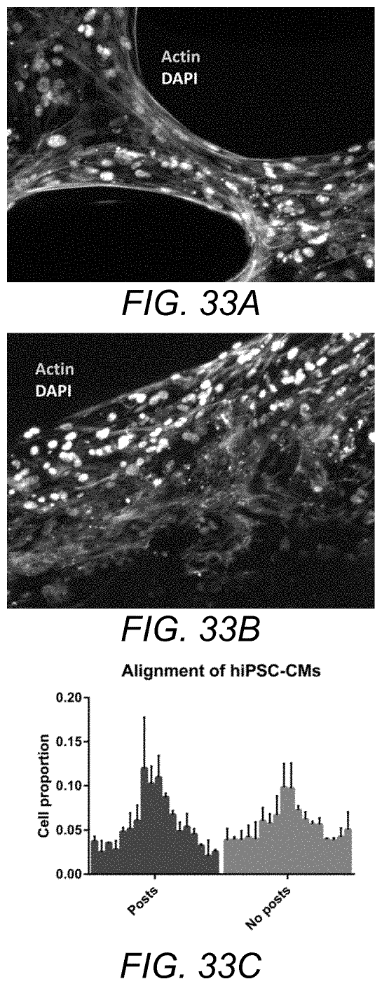

FIGS. 32A-34B represent items obtained from additional studies performed on a co-culture of hiPSC-CMs and human embryonic stem cell-derived cardiomyocytes (hESC-CMs), together referred to as human pluripotent stem cells (hPSCs), in hydrogel-encapsulated tissue within the cell suspension region 18 of the microfluidic device 10 as disclosed herein. Specifically, hPSC-CMs were encapsulated with CFs in a 3D biomimetic hydrogel, and the cell:hydrogel suspension was injected into the microfluidic device 10. After culture within the device for 14 days, the co-cultured cells formed an anisotropic tissue with high expression of connexin 43 (CX43) and sarcomeric alpha actinin. FIGS. 32A-32C provide a characterization of the cardiac cell population from differentiation of hiPSCs. FIG. 32A is an immunofluorescent stained image of sarcomeric alpha actinin (α-actinin, lighter gray) and vimentin (medium gray) to identify populations of cardiomyocytes and cardiac fibroblasts, respectively. FIG. 32B is an immunofluorescent stained image of sarcomeric alpha actinin (lighter gray) and connexin 43 (CX43, medium gray) of hiPSC-CM population. FIG. 32C is a magnified image of the immunofluorescent stained image of FIG. 32B.

In comparison, a device without the staggered elliptical microposts within the channel (referred to as “no posts”), was used to house the cardiac tissue for the same period, and the resulting tissue was compared to that formed in the proposed microfluidic device 10. It was found that the cardiac tissues within the microfluidic device 10 with the vertical posts 22 were more aligned, elongated, and exhibited a high degree of maturation, through gene and protein level expression, than in the devices without the posts. The culture of cells within a 3D hydrogel around the precisely spaced elliptical vertical posts 22 enabled the creation of a highly aligned cardiac tissue, derived from human stem cells, with a maturation state more physiologically relevant to the human myocardium than hPSC-CMs cultured in typical monolayer format.

FIGS. 33A-33C provide an assessment of the alignment of hiPSC-derived cardiac tissues formed within devices with and without the vertical posts 22. FIG. 33A is an Actin (medium gray) stained image of cardiac tissues within the microfluidic device 10 including vertical posts 22. FIG. 33B is an Actin (medium gray) stained image of cardiac tissues within devices without posts. FIG. 33C is a plot quantifying cell alignment around an alignment axis, demonstrating a higher proportion of cells aligned along the alignment axis (i.e. at 0°) in cardiac tissues formed in the microfluidic device 10 with vertical posts 22 than in devices without posts.

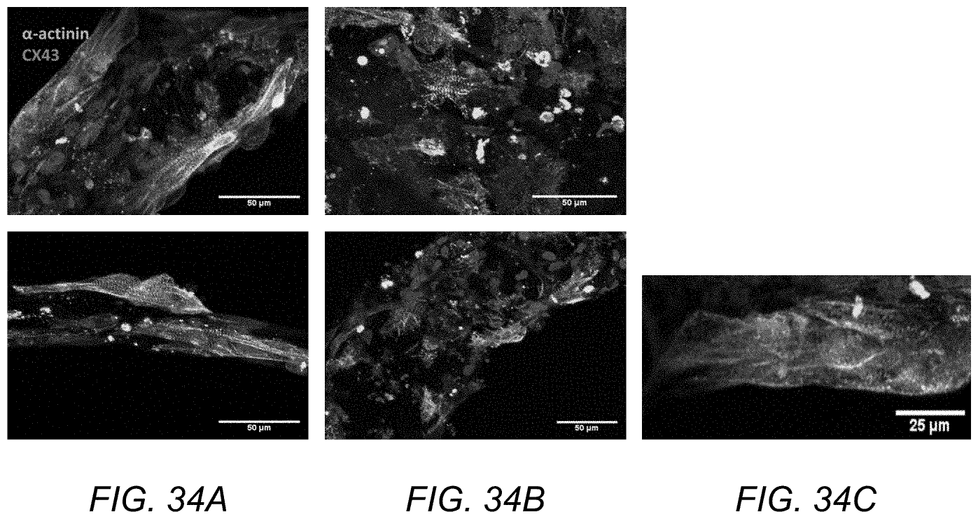

FIGS. 34A-34C provide images of expression of cardiac markers of hiPSC-derived cardiac tissues inside the microfluidic device 10 and devices without posts. FIG. 34A is an immunofluorescent stained image of sarcomeric alpha actinin (lighter gray) and CX43 (medium gray) of cardiac tissues formed after 2 weeks in the microfluidic device 10 with vertical posts 22. FIG. 34B is an immunofluorescent stained image of sarcomeric alpha actinin (lighter gray) and CX43 (medium gray) of cardiac tissues formed after 2 weeks in devices without posts. FIG. 34C is a magnified image of the immunofluorescent stained image of FIG. 34A.

Although cardiac cells were used to show the success of the platform, cells of other types such as neural, embryonic stem, or skeletal cells could be used in microfluidic devices 10 as disclosed herein. As will be recognized by one skilled in the art, culture details may vary depending on the types of cells used.

Embodiments disclosed herein may provide one or more of the following technical benefits. Microfluidic devices 10 disclosed herein integrate highly organized microposts (e.g., the vertical posts 22) with a cell suspension region 18 to induce 3D cellular and tissue-level alignment similar, for instance, to the architecture of the native human myocardium in a microscale platform. Despite the recent emergence of in vitro heart-on-a-chip technologies, most of the previously reported platforms have been ill-suited for producing highly aligned cardiac tissue. Microfluidic devices 10 disclosed herein also enable enhanced sample collection (i.e., cells) from the cell suspension region 18 by providing separate sample extraction ports 40, 42 downstream of two lateral portions of the cell suspension region 18 to enable performance downstream genetic analyses. Such analyses are necessary in defining the underlying mechanisms of diseased cardiac tissue at molecular level or other types of diseases depending on the type of tissue (e.g., brain or skeletal tissue). The ability to dissociate and collect the hydrogel-encapsulated tissue to perform genetic analyses on the cultured cells is highly beneficial. Additionally, microfluidic devices 10 disclosed herein enable sufficient collection of media samples from the first fluid channel 44 and the second fluid channel 46 for analysis on the secreted cytokines/proteins using standard assays such as ELISA or LCMS. Moreover, microfluidic devices 10 disclosed herein provide the ability to create specific injury and/or disease models through manipulation of the precisely controlled factors within the tissue microenvironment. This platform allows for specific and directed application of insults, such as drugs or environmental factors, on the encapsulated tissue (e.g., cardiac, brain, or skeletal tissue). Additionally, the platform allows for continuous, real-time monitoring of the cardiac tissue in both the healthy and injured/diseased state.

Although various examples herein are directed to cardiac tissue, it is to be appreciated that the disclosure is not so limited, and that any suitable types of cells may be cultured in a cell suspension region 18 of a microfluidic device 10 as disclosed herein.

Those skilled in the art will recognize improvements and modifications to the preferred embodiments of the present disclosure. All such improvements and modifications are considered within the scope of the concepts disclosed herein and the claims that follow.

1. A microfluidic device comprising:
a cell suspension region;
a plurality of linear arrays of vertical posts arranged within the cell suspension region;
at least one hydrogel insertion port arranged upstream of the cell suspension region;
at least one sample extraction port arranged downstream of the cell suspension region;
a first fluid channel arranged proximate to the cell suspension region;
a first fluid-permeable boundary wall arranged between the first fluid channel and the cell suspension region, and forming a first lateral boundary of the cell suspension region;
a second fluid channel arranged proximate to the cell suspension region;
a second fluid-permeable boundary wall arranged between the second fluid channel and the cell suspension region, and forming a second lateral boundary of the cell suspension region;
a first fluid channel inlet port and a first fluid channel outlet port in fluid communication with the first fluid channel; and
a second fluid channel inlet port and a second fluid channel outlet port in fluid communication with the second fluid channel;
wherein the cell suspension region is enclosed from above and below.

2. The microfluidic device of claim 1, wherein the cell suspension region contains a three-dimensional (3D) hydrogel matrix having cells embedded therein and arranged in contact with the plurality of linear arrays of vertical posts.

3. The microfluidic device of claim 2, wherein the cells comprise at least one of: animal- or human-derived cardiac cells, animal- or human-derived neural cells, or animal- or human-derived skeletal muscle cells.

4. The microfluidic device of claim 1, wherein each vertical post of the plurality of linear arrays of vertical posts includes a tapered leading edge and a tapered trailing edge.

5. The microfluidic device of claim 4, wherein each vertical post of the plurality of linear arrays of vertical posts includes a length, from the tapered leading edge to the tapered trailing edge, in a range of from 200 microns to 500 microns.

6. The microfluidic device of claim 1, wherein vertical posts of the plurality of linear arrays of vertical posts are spaced apart from adjacent vertical posts by a lengthwise dimension in a range of from 100 to 300 microns, and by a widthwise dimension in a range of from 100 to 300 microns.

7. The microfluidic device of claim 1, wherein the first fluid-permeable boundary wall comprises a first array of trapezoidal posts, and the second fluid-permeable boundary wall comprises a second array of trapezoidal posts.

8. The microfluidic device of claim 7, wherein:
the first array of trapezoidal posts comprises a first plurality of linearly arranged trapezoidal posts, with each trapezoidal post of the first plurality of linearly arranged trapezoidal posts comprising a short end arranged closer to the cell suspension region than to the first fluid channel; and
the second array of trapezoidal posts comprises a second plurality of linearly arranged trapezoidal posts, with each trapezoidal post of the second plurality of linearly arranged trapezoidal posts comprising a short end arranged closer to the cell suspension region than to the second fluid channel.

9. The microfluidic device of claim 1, wherein the at least one sample extraction port comprises a first sample extraction port and a second sample extraction ports that are laterally offset relative to one another.

10. The microfluidic device of claim 9, further comprising:
a first sample extraction channel arranged between the cell suspension region and the first sample extraction port; and
a second sample extraction channel arranged between the cell suspension region and the second sample extraction port;
wherein the first sample extraction channel is laterally offset relative to the second sample extraction channel.

11. The microfluidic device of claim 1, wherein:
the at least one hydrogel insertion port comprises a first hydrogel insertion port and a second hydrogel insertion port that are laterally offset relative to one another; and
the microfluidic device further comprises a first hydrogel insertion channel arranged between the first hydrogel insertion port and the cell suspension region, and a second hydrogel insertion channel arranged between the second hydrogel insertion port and the cell suspension region, wherein the first hydrogel insertion channel is laterally offset relative to the second hydrogel insertion channel.

12. The microfluidic device of claim 1, further comprising endothelial cells seeded in at least one of the first fluid channel or the second fluid channel.

13. The microfluidic device of claim 1, wherein one or more of the first fluid channel, the second fluid channel, or the cell suspension region comprises a height dimension and/or width dimension of less than 500 microns.

14. The microfluidic device of claim 1, further comprising at least one electrode arranged in conductive electrical communication with the cell suspension region.

15. A microfluidic device comprising:
a cell suspension region;
at least one hydrogel insertion port arranged upstream of the cell suspension region;
a plurality of sample extraction ports arranged downstream of and in fluid communication with the cell suspension region, wherein each sample extraction port of the plurality of sample extraction ports is laterally offset relative to at least one other sample extraction port of the plurality of sample extraction ports;
a first fluid channel arranged proximate to the cell suspension region;
a first fluid-permeable boundary wall arranged between the first fluid channel and the cell suspension region, and forming a first lateral boundary of the cell suspension region;
a second fluid channel arranged proximate to the cell suspension region;
a second fluid-permeable boundary wall arranged between the second fluid channel and the cell suspension region, and forming a second lateral boundary of the cell suspension region;
a first fluid channel inlet port and a first fluid channel outlet port in fluid communication with the first fluid channel; and
a second fluid channel inlet port and a second fluid channel outlet port in fluid communication with the second fluid channel;
wherein the cell suspension region is enclosed from above and below.

16. The microfluidic device of claim 15, further comprising a plurality of linear arrays of vertical posts arranged within the cell suspension region.

17. The microfluidic device of claim 16, wherein each vertical post of the plurality of linear arrays of vertical posts includes a tapered leading edge and a tapered trailing edge.

18. The microfluidic device of claim 17, wherein each vertical post of the plurality of linear arrays of vertical posts includes a length, from the tapered leading edge to the tapered trailing edge, in a range of from 200 microns to 500 microns.

19. The microfluidic device of claim 16, wherein vertical posts of the plurality of linear arrays of vertical posts are spaced apart from adjacent vertical posts by a lengthwise dimension in a range of from 100 to 300 microns, and by a widthwise dimension in a range of from 100 to 300 microns.

20. The microfluidic device of claim 16, wherein the cell suspension region contains a three-dimensional (3D) hydrogel matrix having cells embedded therein and arranged in contact with the plurality of linear arrays of vertical posts.

21. The microfluidic device of claim 15, wherein the first fluid-permeable boundary wall comprises a first array of trapezoidal posts, and the second fluid-permeable boundary wall comprises a second array of trapezoidal posts.

22. The microfluidic device of claim 21, wherein:
the first array of trapezoidal posts comprises a first plurality of linearly arranged trapezoidal posts, with each trapezoidal post of the first plurality of linearly arranged trapezoidal posts comprising a short end arranged closer to the cell suspension region than to the first fluid channel; and
the second array of trapezoidal posts comprises a second plurality of linearly arranged trapezoidal posts, with each trapezoidal post of the second plurality of linearly arranged trapezoidal posts comprising a short end arranged closer to the cell suspension region than to the second fluid channel.

23. The microfluidic device of claim 15, wherein:
the plurality of sample extraction ports comprises a first sample extraction port and a second sample extraction port; and
the microfluidic device further comprises a first sample extraction channel arranged between the cell suspension region and the first sample extraction port, and a second sample extraction channel arranged between the cell suspension region and the second sample extraction port, wherein the first sample extraction channel is laterally offset relative to the second sample extraction channel.

24. The microfluidic device of claim 15, wherein:
the at least one hydrogel insertion port comprises a first hydrogel insertion port and a second hydrogel insertion port that are laterally offset relative to one another; and
the microfluidic device further comprises a first hydrogel insertion channel arranged between the first hydrogel insertion port and the cell suspension region, and a second hydrogel insertion channel arranged between the second hydrogel insertion port and the cell suspension region, wherein the first hydrogel insertion channel is laterally offset relative to the second hydrogel insertion channel.

25. The microfluidic device of claim 15, further comprising endothelial cells seeded in at least one of the first fluid channel or the second fluid channel.

26. The microfluidic device of claim 15, wherein one or more of the first fluid channel, the second fluid channel, or the cell suspension region comprises a height dimension and/or width dimension of less than 500 microns.

27. The microfluidic device of claim 15, further comprising at least one electrode arranged in conductive electrical communication with the cell suspension region.

28.-36. (canceled)
